(19) United States
(12) Patent Application Publication
(10) Pub. No.: US 2012/0039832 A1
(43) Pub. Date: Feb. 16, 2012

(54) TOPICAL COMPOSITIONS AND METHODS

(76) Inventor: Eric H. Barry, North East, PA (US)

(21) Appl. No.: 13/196,420

(22) Filed: Aug. 2, 2011

Related U.S. Application Data
(60) Provisional application No. 61/372,234, filed on Aug. 10, 2010.

(51) Int. Cl. A61K 8/92; A61Q 15/00

(52) U.S. Cl. 424/65

(57) ABSTRACT

Topical compositions include a vegetable oil, a natural wax, and activated charcoal. In further examples, methods of reducing or eliminating body odor comprise the steps of applying a topical composition comprising: a vegetable oil; a natural wax; and activated charcoal. In still further examples, methods of making a topical application device comprise the steps of providing a topical composition comprising vegetable oil, natural wax and activated charcoal. The methods further include the steps of charging a dispensing device with the topical composition, wherein the dispensing device is configured to apply a quantity of the topical composition at a desired location.

### CROSS-REFERENCE TO RELATED APPLICATIONS

This application claims the benefit of U.S. Provisional Application No. 61/372,234, filed Aug. 10, 2010, the entire disclosure of which is hereby incorporated herein by reference.

### TECHNICAL FIELD

This application relates generally to topical compositions and methods and, more particularly, to topical compositions methods of making and using topical compositions with a vegetable oil; a natural wax; and activated charcoal.

### BACKGROUND

Known deodorants and antiperspirants attempt to control odor through a variety of means. Some deodorant compositions attempt to mask odor through perfumes and/or fragrances. Other deodorant compositions may employ antibacterial compounds that help destroy and/or control the amount of bacteria present on the skin, thereby minimizing odor produced via bacterial metabolism of the skin secretions. Antiperspirants, on their own or in combination with a deodorant, attempt to prevent odor by preventing sweating, thus eliminating perspiration, by affecting sweat glands. Many antiperspirants employ aluminum-based complexes, which are designed to react with ions found in perspiration to form a plug in the duct of the sweat gland.

There remains a continuing need for an improved odor and moisture absorbing compositions. More particularly, there is a need for compositions that have both deodorizing and antiperspirant properties that do not utilize aluminum-based compounds.

### SUMMARY

The following presents a simplified summary of the disclosure in order to provide a basic understanding of some example aspects described in the detailed description.

In accordance with one aspect, a topical composition includes a vegetable oil, a natural wax, and activated charcoal.

In accordance with another aspect, a method of reducing or eliminating body odor comprises the step of applying a topical composition comprising: a vegetable oil; a natural wax; and activated charcoal.

In accordance with another aspect, a method of making a topical application device comprises the step of providing a topical composition comprising vegetable oil, natural wax and activated charcoal. The method further includes the step of charging a dispensing device with the topical composition, wherein the dispensing device is configured to apply a quantity of the topical composition at a desired location.

### BRIEF DESCRIPTION OF THE DRAWINGS

The foregoing and other aspects of the present invention will become apparent to those skilled in the art to which the present invention relates upon reading the following description with reference to the accompanying drawings, in which:

FIG. 1 illustrates an example step of providing a topical composition including melting a quantity of natural wax;

FIG. 2 illustrates another example step of providing a topical composition including adding a quantity of vegetable oil to the melted wax from FIG. 1;

FIG. 3 illustrates another example step of providing a topical composition including adding a quantity of activated charcoal to the melted wax and vegetable oil of FIG. 2;

FIG. 4 illustrates another example step of providing a topical composition including providing another additive such as one or more fragrances and/or one or more colorants;

FIG. 5 illustrates an example step of making a topical application device comprising the step charging a product reservoir of a dispensing device with the topical composition of FIG. 3 or FIG. 4;

FIG. 6 illustrates an example of the dispensing device immediately after fully charging with the topical composition of FIG. 3 or FIG. 4;

FIG. 7 illustrates the topical application device with the topical composition of FIG. 6 being cooled to a solid;

FIG. 8 is an example of the topical application device of FIG. 7 ready for use to reduce or eliminate body odor;

FIG. 9 illustrates the topical application device of FIG. 8 with the protective cap removed and the topical composition being exposed for subsequent application; and

FIG. 10 illustrates applying a quantity of the topical composition to a desired location with the topical application device.

### DETAILED DESCRIPTION

Example embodiments that incorporate one or more aspects of the present invention are described and illustrated in the drawings. These illustrated examples are not intended to be a limitation on the present invention. For example, one or more aspects of the present invention can be utilized in other embodiments and even other types of devices. Moreover, certain terminology is used herein for convenience only and is not to be taken as a limitation on the present invention.

Some topical compositions of the present disclosure consist essentially of a vegetable oil; a natural wax; and activated charcoal. In one example, some topical compositions can consist essentially (or entirely) of these three ingredients with little if any additional ingredients. In further examples, topical compositions of the present disclosure include a vegetable oil; a natural wax; and activated charcoal with other possible ingredients. For instance, as discussed below, the topical compositions can include one or more additives, such as one or more fragrances and/or one or more colorants.

Each topical composition of the present disclosure includes a vegetable oil. For purposes of this application, a vegetable oil is considered a lipid-based material derived from plants. At room temperature (e.g., 22° C.), vegetable oils can be a liquid although the vegetable oils can be a mixture of liquids and solids or other examples. Example vegetable oils can include one or more of olive oil, soya oil, peanut oil, avocado oil, cocoa butter, sunflower seed oil, jojoba oil, cottonseed oil, sunflower oil, palm oil, palm seed oil, linseed oil, almond oil, castor oil, corn oil, rapeseed oil, sesame seed oil, safflower oil, wheat germ oil, peach seed oil, coconut oil although other vegetable oils may be used in further examples.

In one illustrative example, the vegetable oil comprises olive oil, such as non-virgin olive oil. Olive oil is readily available, for example, by mechanical or chemical extraction from the fruit of an olive tree. For purposes of this application, virgin olive oil is considered olive oil produced by the use of physical extraction without chemical treatment. Non-virgin olive oil, for purposes of this application, is olive oil produced with refining techniques that involve non-physical extraction, either alone or in combination with physical extraction techniques. For example, non-virgin olive oil can include olive oils that are refined and/or a blend of refined and virgin olive oils.

Although fatty acid composition varies widely based on the variety of plants used, the major fatty acids in olive oil are oleic acid (C18:1, 55-83%) and linoleic acid (C18:2, 3.5-21%) and palmitic acid (C16:0, 7.5-20%). Olive oil is reported to have a density ranging from 0.8 g/mL to 0.92 g/mL, depending on the precise composition.

In another illustrative example, the vegetable oil can comprise soybean oil. Soybean oil is extracted from the seeds of the soybean and includes mostly C-18 saturated and unsaturated fatty acids, including α-linoleic acid (7-10%, C18:3), linoleic acid (˜51%, C18:2), and oleic acid (˜23%, C18:1) as the major constituents.

Each topical composition of the present disclosure also includes a natural wax. Natural waxes comprise waxes from an animal, vegetable, mineral or hydrocarbon origin although other natural origins may be provided in further examples. For instance, the topical composition can comprise a natural wax with an animal origin such as beeswax, woolwax, spermateci and bear fat, shellac wax, and the like. In further examples, the topical composition can comprise a natural wax with a vegetable origin such as shea butter, cocoa butter, Japan wax, esparto grass wax, cork wax, rice shoot wax, ouricury wax, montan wax, sunflower wax, ceresine wax, sugar cane wax, carnauba wax, candelilla wax, lanolin, fruit-derived waxes, such as orange wax, lemon wax, grapefruit wax and bayberry wax, and the like. In still further examples the topical composition can comprise a natural wax comprising a mineral wax such as ceresine and ozokerite waxes and hydrocarbon waxes such as paraffin.

In one illustrative example, the topical composition includes a natural wax comprising beeswax. For purposes of this application, beeswax is a natural wax produced by honeybees to form the comb of the beehive. Beeswax is secreted by glands on the bee's abdomen as a byproduct of honey consumption by the bee. In the hive, it is molded into cells for pollen and honey storage as well as for raising brood. Beeswax is comprised of mainly of fatty acids and various long chain alcohols and has a melting point range of about 62 to 64° C. One advantage of beeswax for use in the present application is the inclusion of its natural color and scent. Most beeswax is off white to yellow in color. Both the natural color and scent of beeswax depends on the types of plants from which pollen was collected by the bees and often varies regionally.

Before use in the present application, the beeswax may be melted and cleaned through a process called refining. When honeybees in the hive use the beeswax, the beeswax can be stained by the color of stored pollen or stored honey. Various kinds of debris, including dead bees, honey, pollen, and cocoon debris also collect in the wax. In order to obtain cleaner, more usable wax, the beeswax may be refined prior to incorporation as an optional component of the topical composition. One common method for refining beeswax includes melting the beeswax slowly in water to just above its melting point. Some of the debris will collect on the top and some will collect at the bottom. Upon cooling, the top and bottom portions of the cooled wax cake can be removed (for example, by scraping) to eliminate the debris and obtain a clean, refined beeswax product. Refining can also be accomplished chemically and may include a step of bleaching to obtain a lighter product. Both beeswax and refined beeswax are available for purchase from a variety of commercial sources.

In another illustrative example, the natural wax can comprise paraffin (paraffin wax). Paraffin is a white, odorless, tasteless, waxy solid derived from a mixture of hydrocarbons. Its melting point is between about 46 and 68° C.

Each topical composition of the present disclosure also includes activated charcoal. Activated charcoal is also known as activated carbon or activated coal. Activated charcoal can take the form of carbon that has been processed to provide a very large surface area that is available for adsorption. For example, the activated charcoal can include a microporous structure providing extensive adsorptive properties that, once activated, can adsorb a vast variety of organic compounds, including those that can contribute to the production of human or animal malodors. The important characteristics of the activated carbon include available surface area, pore size and size distribution, surface chemistry, particle size, and particle density. A particle of activated carbon is typically spherical with substantial adsorbent surface area and pore cavity. On example particle size of activated carbon that may be used in the topical composition can range in size from about 3 microns to about 500 microns, such as from about 5 microns to about 250 microns although other particle sizes may be incorporated in accordance with further examples of the disclosure. The internal surface area, which can provide beneficial adsorbent characteristics, typically ranges from about 100 to about 1000 m²/gm. Activated charcoal can be obtained from a variety of commercial sources.

As mentioned previously, the topical composition may optionally include ingredients other than a vegetable oil; a natural wax; and activated charcoal. For example, some examples can include an addition of fragrances and/or colorants. Such fragrances and or colorants can provide, such as modify, the existing fragrance and/or color of the topical composition based on the particular desired product features. In one example, natural colorants and/or fragrances may be used although artificial colors and fragrances may also be employed in further examples.

In one particular embodiment, the topical composition can comprise a vegetable oil comprising olive oil, such as non-virgin olive oil. The topical composition can further include a natural wax comprising a beeswax, such as a refined beeswax. The topical composition can further include activated charcoal. The vegetable oil (e.g., non-virgin olive oil), natural wax (e.g., refined beeswax), and activated charcoal can be provided in concentrations sufficient to produce a solid (e.g., a stiff or soft solid) at 22° C. As discussed more fully below, the solid can be formed as a bar for charging in a dispensing device.

In another example, the topical composition can comprise a natural wax, (e.g., refined beeswax) at about 50 to about 63% w/w, such as about 52% w/w. In addition or alternatively, the topical composition can comprise a vegetable oil (e.g., non-virgin olive oil) present at about 35 to about 49% w/w, such as about 47% w/w. In addition or alternatively, the topical composition can comprise activated charcoal present at about 0.01 to about 3% w/w, such as about 1% w/w. The following are nonlimiting example topical compositions in accordance with aspects of the present disclosure.

### Example 1

| Ingredient | Amount | | % w/w |
|---|---|---|---|
| refined beeswax | 25.5 | g | 60.0 |
| non-virgin olive oil | 18 | mL* | 38.2 |
| activated charcoal | 0.76 | g | 1.8 |

*assumes a density for non-virgin olive oil of 0.9 g/mL

### Example 2

| Ingredient | Amount | | % w/w |
|---|---|---|---|
| refined beeswax | 85.1 | g | 52.6 |
| non-virgin olive oil | 63 | mL* | 47.3 |
| activated charcoal | 0.19 | g | 0.1 |

*assumes a density for non-virgin olive oil of 0.9 g/mL

### Example 3

| Ingredient | Amount | | % w/w |
|---|---|---|---|
| refined beeswax | 85.1 | g | 52.1 |
| non-virgin olive oil | 63 | mL* | 46.9 |
| activated charcoal | 1.52 | g | 0.9 |

*assumes a density for non-virgin olive oil of 0.9 g/mL

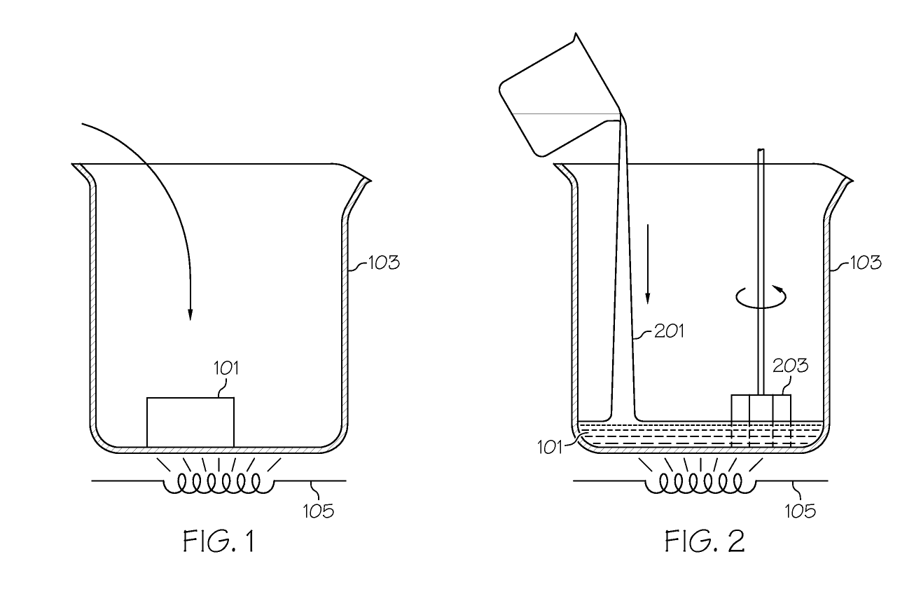

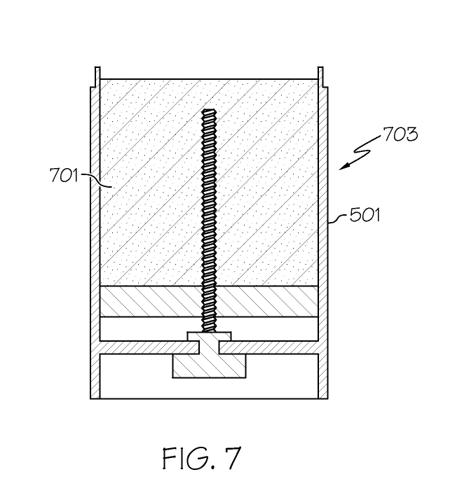

Methods of making the topical application device 703 (shown in FIG. 7) will not be described, for example, with reference to FIGS. 1-7. As shown in FIGS. 1-3, the method can begin with the step of providing a topical composition comprising a natural wax 101, a vegetable oil 201, and activated charcoal 301 in accordance with any of the previous/mentioned examples.

As shown in FIG. 1, the method can include the step of providing a quantity of the natural wax 101 (e.g., refined beeswax) in a containment area of a container 103. A heater 105 can then be activated to melt the natural wax 101, for example, to a temperature of from about 65° C. to about 85° C. Regarding the melting step of the disclosed methods, the temperature to which the natural wax shall be melted will depend on the particular natural wax used in the composition. For example, the melting point of beeswax is expected to be approximately 62 to 64° C. and the melting point of paraffin is expected to be between approximately 46 and 68° C. The natural wax should not be overheated, as discoloration and product breakdown may begin to occur. In one example, the natural wax may be heated to approximately 5 to 10° C. above its melting point.

As shown in FIG. 2, the method can also include the step of providing a quantity of the vegetable oil 201 (e.g., non-virgin olive oil) in the containment area of the container 103. As shown, a mixing device 203 can be used to stir together the melted wax 101 and the vegetable oil 201. In one example, the melted wax 101 and the vegetable oil 201 can be stirred to the extent that a substantially homogeneous mixture is achieved. When adding the vegetable oil 201 and/or during stirring with the mixing device 203, the heater 105 can continue to apply heat to the container 103 such that the mixture 101, 201 at least substantially maintains a liquid state.

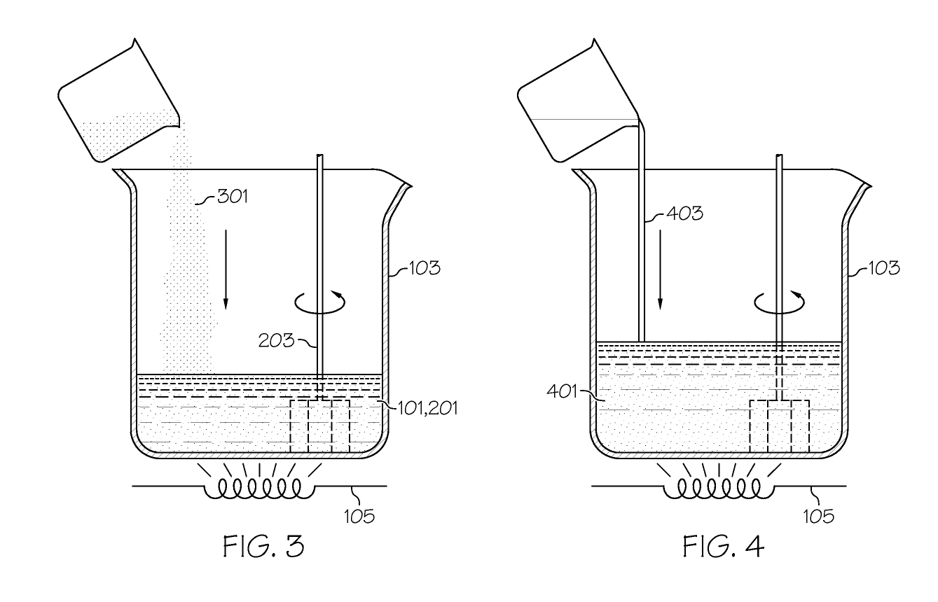

As shown in FIG. 3, the method can also include the step of providing a quantity of activated charcoal 301 to the mixture 101, 201 of the melted wax 101 and the vegetable oil 201. The mixing device 203 can continue to mix the ingredients together to obtain a substantially homogenous mixture 401 of melted wax 101, vegetable oil 201 and activated charcoal 301. When adding the activated charcoal 301 and/or during stirring with the mixing device 203, the heater 105 can continue to apply heat to the container 103 such that the mixture 401 maintains a substantially liquid state.

Providing the natural wax 101, vegetable oil 201 and activated charcoal 301 may be provided in any order and the natural wax may be melted at any time during the process of obtaining the mixture 401. For example, the vegetable oil 201 may be provided before adding the natural wax. The heat can then be provided to melt the wax while mixing the melted wax together with the vegetable oil. Then the activated charcoal may be added. In another example, the activated charcoal may be provided first, or be mixed together with one of the natural wax and/or the vegetable oil before completing the mixture.

In further examples, the natural wax is melted and then the vegetable oil is added to the natural wax to obtain a first homogeneous mixture. The activated charcoal can then be added to the natural wax/vegetable oil mixture to obtain a second homogeneous mixture. In still another embodiment, a first homogeneous mixture may be formed by mixing the vegetable oil and activated charcoal, which is then added to the melted natural wax to obtain a second homogeneous mixture. In yet another embodiment, the natural wax may first be melted, to which the activated charcoal may be added to obtain a first homogeneous mixture, followed by addition of the vegetable oil to obtain a second homogeneous mixture.

As shown in FIG. 4, the method may further include the optional step of providing an additive 403 such as one or more fragrances and/or one or more colorants. Such additive, if provided, can be introduced at the beginning, during or at the end of mixing together the natural wax, vegetable oil and activated charcoal. As shown, the mixing device 203 may be used to mix the mixture 401 of melted wax, vegetable oil and activated charcoal together with the additive 403 to obtain a substantially homogenous mixture. Moreover, the heater 105 can be used to maintain the mixture in a substantially liquid state.

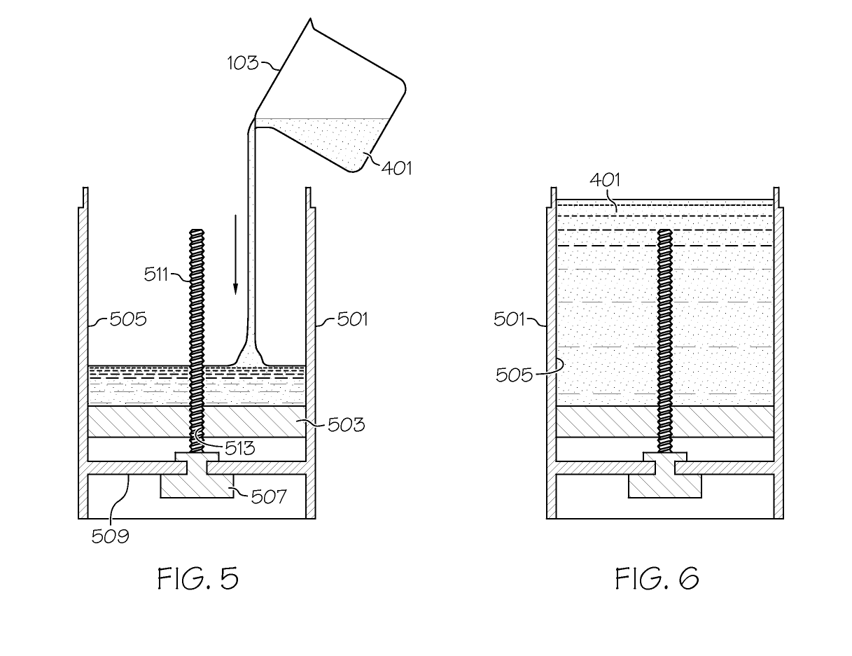

FIG. 5 illustrates an example step of charging a dispensing device 501 with the mixture 401. As shown, the dispensing device 501 can include a piston 503 slidingly received within an interior area 505 of the dispensing device 501. A knob 507 may be rotatably coupled to a lower wall 509. A threaded shaft 511 can be threadingly received within a threaded aperture 513 of the piston 503. The knob 507 can be rotated such that the piston 503 is in the fully retracted position to maximize the interior area 505 that may receive the mixture 401.

As further illustrated in FIG. 5, the liquid nature of the mixture 401 can allow the mixture to be poured into the interior area 505 to charge the interior area 505 with a quantity of the mixture 401 as shown in FIG. 6.

As shown in FIG. 7, the method can further include the step of cooling the mixture 401 wherein the topical composition including the natural wax, vegetable oil and activated charcoal cools to form a solid topical composition 701. Various methods may be used to cool the mixture. For example, active cooling methods may involve an external cooling source, such as use of a refrigeration or freezing unit to quickly reduce the temperature of the mixture. Passive cooling methods allow for slower, more controlled cooling of the mixture. Passive cooling is shown to transform the mixture from the liquid state shown in FIG. 6 to the solid state shown in FIG. 7. In some examples, slower, more controlled cooling may be employed to preserve the texture and/or color of the solid topical composition.

Once cooled the solid topical composition 701 can form firm stick or soft solid while being effectively charged within the dispensing device 501 forms a topical application device 703.

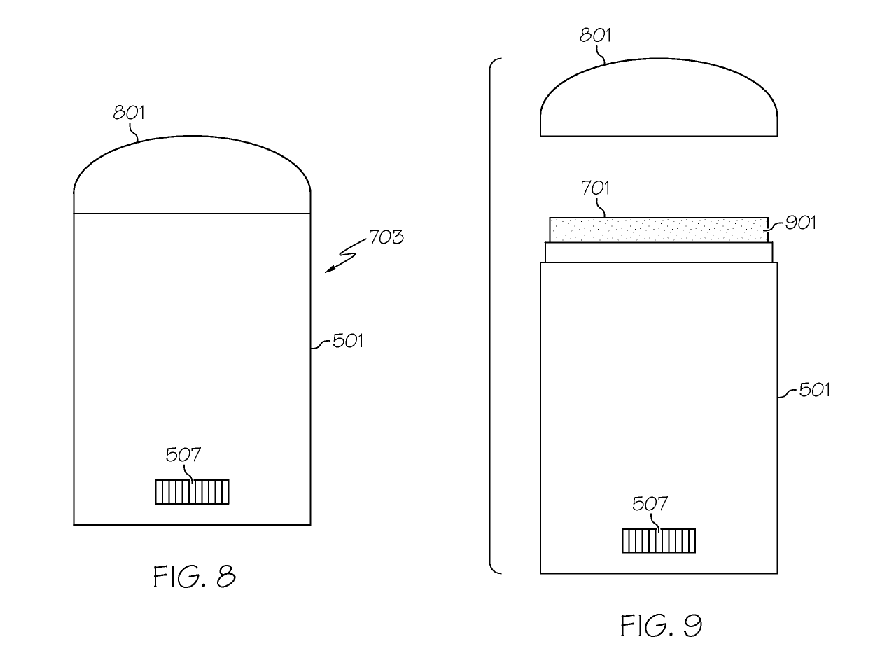

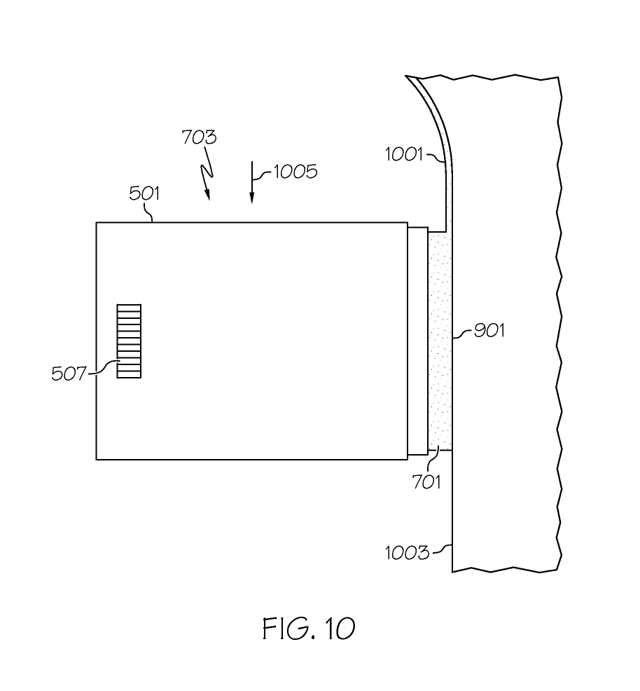

FIGS. 8-10 illustrate a method of reducing or eliminating body odor with the topical application device 703. As shown in FIG. 8, the topical application device 703 may initially be shipped with an optional protective cap 801. As shown in FIG. 9, the cap 801 can be removed and the knob 507 can be adjusted to extend the piston 503 together with the solid topical composition 701, thereby exposing an end portion 901. As shown in FIG. 10, a quantity 1001 of the solid topical composition 701 can be applied to a desired location, such as the skin 1003 of an animal or human. The solid topical composition 701 can be applied by pressing the end portion 901 against the skin 1003 and moving the topical application device 703 in direction 1005. As the exposed end portion 901 is exhausted, the knob 507 may again be adjusted to expose additional portions of the solid topical composition 701 for dispensing.

In one example, the solid topical composition 701 can be applied to the human skin to obtain deodorant and/or antiperspirant properties before or during a time of need. The disclosed composition can be applied to any area in need of such deodorant and/or antiperspirant properties.

The invention has been described with reference to the example embodiments described above. Modifications and alterations will occur to others upon a reading and understanding of this specification. Examples embodiments incorporating one or more aspects of the invention are intended to include all such modifications and alterations insofar as they come within the scope of the appended claims.

What is claimed is:

1. A topical composition comprising:
a vegetable oil;
a natural wax; and
activated charcoal.

2. The composition according to claim 1, further comprising one or more fragrances and/or one or more colorants.

3. The composition according to claim 1, wherein the natural wax is paraffin.

4. The composition according to claim 1, wherein the natural wax is beeswax.

5. The composition according to claim 4, wherein the beeswax is refined beeswax.

6. The composition according to claim 1, wherein the vegetable oil is soybean oil.

7. The composition according to claim 1, wherein the vegetable oil is olive oil.

8. The composition according to claim 7, wherein the olive oil is non-virgin olive oil.

9. The composition according to claim 1, wherein the vegetable oil, the natural wax and the activated charcoal are present in concentrations sufficient to produce a solid at 22° C.

10. The composition according to claim 1, wherein the natural wax is present at about 50 to about 63% w/w, the vegetable oil is present at about 35 to about 49% w/w, and the activated charcoal is present at about 0.01 to about 3% w/w.

11. The composition according to claim 10, wherein the natural wax is present at about 52% w/w, the vegetable oil is present at about 47% w/w, and the activated charcoal is present at about 1% w/w.

12. A method of reducing or eliminating body odor comprising the step of applying a topical composition comprising: a vegetable oil; a natural wax; and activated charcoal.

13. The method according to claim 12, wherein the vegetable oil is olive oil and the natural wax is beeswax.

14. A method of making a topical application device comprising the steps of:
a) providing a topical composition comprising vegetable oil, natural wax and activated charcoal; and
b) charging a dispensing device with the topical composition, wherein the dispensing device is configured to apply a quantity of the topical composition at a desired location.

15. The method according to claim 14, wherein the step of providing the topical composition includes the steps of melting the wax, adding the vegetable oil to the melted wax and adding the activated charcoal to the melted wax.

16. The method of claim 15, wherein the step of melting the wax includes heating the wax to a temperature of from about 65° C. to about 85° C.

17. The method according to claim 15, wherein the step of providing the topical composition includes the step of mixing the melted wax, the vegetable oil and the activated charcoal.

18. The method according to claim 17, wherein the step of mixing provides a substantially homogenous mixture of melted wax, vegetable oil, and activated charcoal.

19. The method according to claim 15, wherein the topical composition is cooled to form a solid topical composition that is charged in the dispensing device.

20. A method of reducing or eliminating body odor with the topical application device made in accordance with claim 14, comprising the steps of:
applying a quantity of the topical composition to a desired location with the topical application device.
